(19) United States
(12) Patent Application Publication
(10) Pub. No.: US 2010/0008864 A1
(43) Pub. Date: Jan. 14, 2010

(54) AROMATIC MULTIMERS

(76) Inventors: Andreas Meijer, Oslo (NO); Harry John Wadsworth, Herts (GB); Ian Martin Newington, Amersham (GB); Clare L. Jones, Harrow (GB); Amanda Ewan, Amersham (GB); Dennis O'Shea, Amersham (GB); Oskar Axelsson, Hagan (NO); Anders Brathe, Oslo (NO); Andreas Olsson, Oslo (NO); John Henrik Johansen, Oslo (NO); Duncan George Wynn, Amersham (GB)

Correspondence Address: GE HEALTHCARE, INC., IP DEPARTMENT 101 CARNEGIE CENTER, PRINCETON, NJ, 08540-6231, US

(21) Appl. No.: 12/499,281

(22) Filed: Jul. 8, 2009

(30) Foreign Application Priority Data
Jul. 10, 2008 (EP) 08012453.0

(51) Int. Cl. A61K 49/10; C07F 15/00

(52) U.S. Cl. 424/9.361; 546/12

(57) ABSTRACT

The present invention relates to novel compounds of formula (I) and (II), compositions comprising compounds of formula (II) and their use as contrast agents in magnetic resonance (MR) imaging (MRI) and MR spectroscopy (MRS).

### FIELD OF THE INVENTION

The present invention relates to novel compounds of formula (I) and (II), compositions comprising compounds of formula (II) and their use as contrast agents in magnetic resonance (MR) imaging (MRI) and MR spectroscopy (MRS).

### BACKGROUND OF THE INVENTION

MR image signal is influenced by a number of parameters that can be divided into two general categories: inherent tissue parameters and user-selectable imaging parameters. Inherent tissue parameters that affect MR signal intensity of a particular tissue are mainly the proton density, i.e. hydrogen nuclei density of that tissue and its inherent T₁ and T₂ relaxation times. Signal intensity is also influenced by other factors such as flow. The contrast between two adjacent tissues, e.g. a tumour and normal tissue depends on the difference in signal between the two tissues. This difference can be maximised by proper use of user-selectable parameters. User-selectable parameters that can affect MR image contrast include choice of pulse sequences, flip angles, echo time, repetition time and use of contrast agents.

Contrast agents work by effecting the T₁, T₂ and/or T₂* relaxation times and thereby influencing the contrast in the images. Information related to perfusion, permeability and cellular density as well as other physiological parameters can be obtained by observing the dynamic behaviour of a contrast agent.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extra cellular space (i.e. the blood and the interstitium) when administered into the vasculature. They are also cleared relatively rapidly from the body. Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

The existent paramagnetic metal chelates that are used as MR contrast agents have a low relaxivity at the 1.5 T magnetic field that is standard in most of today's MR scanners. In 3 T systems which probably will dominate or at least be a substantial fraction of the market in the future, the intrinsic contrast is lower, all T₁ values are higher and the hardware will be faster, so the need for a contrast agent with good performance at 3 T is considerable. In general, the longitudinal relaxivity (r1) of contrast agents falls off at the high magnetic fields of the modern MR scanners, i.e. 1.5 T, 3 T or even higher. This is due to the fast rotational Brownian motion of small molecules in solution which leads to weaker magnetic field coupling of the paramagnetic metal ion to the water molecules than anticipated.

Many attempts have been made to produce contrast agents with high relaxivity by incorporating the paramagnetic metal chelates into larger molecules, such as various polymers. These attempts have been of limited success because of fast internal rotations or segmental motions. Another approach are paramagnetic metal chelates that are bound to or do bind to proteins. However such compounds suffer from pharmacological and pharmacokinetic disadvantages like long excretion time or the risk for interactions with protein bound drugs. Further the leakage through normal endothelium into the interstitium is still substantial.

The problem with the in vivo use of paramagnetic metal ions in a MRI contrast agent is their toxicity and therefore they are provided as complexes with chelating agents which are more stable and less toxic.

For a paramagnetic metal chelate to be useful as a contrast agent in MRI, it is necessary for it to have certain properties. Firstly, it must have high stability because it is important that the complex does not break down in situ and release toxic paramagnetic metal ions into the body.

Secondly, in order for it to be a potent MRI contrast agent, a paramagnetic metal chelate must have high relaxivity. The relaxivity of a MRI contrast agent refers to the amount of increase in signal intensity (i.e. decrease in T₁) that occurs per mole of metal ions. Relaxivity is dependent upon the water exchange kinetics of the complex.

The solubility of the paramagnetic metal chelate in water is also an important factor when they are used as contrast agents for MRI because they are administered to patients in relatively large doses. A highly water-soluble paramagnetic metal chelate requires a lower injection volume, is thus easier to administer to a patient and causes less discomfort.

U.S. Pat. No. 5,820,849 describes chelated complexes attached to globular cascade polymers, however the structures presented therein are not optimized with respect to compactness, rigidity, metal density or a low degree of deformability. It discloses cascade polymer complexes of varying generations, but the structures do not contain any short linker fragments and can not be considered to be rigid as they contain aliphatic linker fragments with very small rotational barriers.

EP 1480979 also discloses complexes attached to globular cascade polymers. The document discloses chelates attached to a core via branching units containing aliphatic segments that obliviate any rigidity imposed to the attached chelates.

U.S. Pat. No. 5,624,901 and U.S. Pat. No. 5,892,029 both describe a class of chelating agents based on 1-hydroxy-2-pyridinone and 3-hydroxy-2-pyridinone moieties which have a substituted carbamoyl group adjacent the hydroxyl or oxo groups of the ring. The compounds are said to be useful as actinide sequestering agents for in vivo use because of their ability to form complexes with actinides. However, it does not refer directly to the complexes which are formed or to any possibility of using them as MRI contrast agents.

U.S. Pat. No. 4,666,927 also relates to hydroxypyridinones. The preferred compounds have an oxo group in either the 2- or the 4-position and a hydroxyl group in the 1- or 3-position. The only other ring substituents are alkyl groups and the compounds are said to be useful as agents for the treatment of general iron overload.

US-A-2003/0095922 relates to complexes formed between gadolinium (III) ions and an organic ligand. The ligand is said to be based on a pyridinone, pyrimidinone or pyridazinone ring system. The exemplified pyridinone compounds are all 3-hydroxy-2-pyridinones with a carbamoyl group in the 4-position of the ring. The compounds are said to be useful as MRI contrast agents and to have high solubility and low toxicity.

Puerta et al, JACS Chem. Comm. 2006, 128, 2222-2223 describes gadolinium chelates of 3-hydroxy-4-pyrones, which are high relaxivity MRI contrast agents with moderate solubility.

US-A-2006/0292079 describes bifunctional chelates based on the ligands 3-hydroxypyridine-2-one, and 5-hydroxy-pyrimidin-4-one. The gadolinium (III) complexes are used as MRI contrast agents.

### DETAILED DESCRIPTION OF THE INVENTION

The present invention provides novel compounds that perform well as MR contrast agents at high magnetic fields, i.e. above 1.5 T, preferably MR tumour contrast agents. The novel compounds are dendrimeric structures that have slowly rotating bonds and tripodal chelates that due to the high rate of the two inner coordination sphere water molecule exchange with bulk water give the compounds high relaxivity. The novel compounds also have improved solubility.

Thus, in a first aspect the invention provides compounds of formula (I)

A-(B-L-R-(L-R-(L-R-(L-R-(L′-X)ᵣ)ᵣ)ᵣ)ᵣ)ₙ (I)

wherein
A denotes a core;
B is the same or different and denotes a moiety that constitutes an obstacle for the rotation of the covalent bond between B and L.
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X is the same or different and denotes a tripodal chelator;
r r is the same or different and denotes the integer 2, 3 or 4; and
n denotes an integer of 3 to 6.

The term “chelator” denotes a chemical entity that binds (complexes) a metal ion to form a chelate. If the metal ion is a paramagnetic metal ion, the chemical entity, i.e. complex formed by said paramagnetic metal ion and said chelator is denoted a “paramagnetic chelate”. The term “tripodal” denotes a chelator that has a three-legged structure. The term “paramagnetic tripodal chelate” denotes a paramagnetic chelate consisting of a tripodal chelator which binds a paramagnetic metal ion to form a paramagnetic tripodal chelate.

A preferred embodiment of a compound of formula (I) is a compound of formula (II)

A-(B-L-R-(L-R-(L-R-(L-R-(L′-X′)ᵣ)ᵣ)ᵣ)ᵣ)ₙ (II)

wherein
A denotes a core;
B is the same or different and denotes a moiety that constitutes an obstacle for the rotation of the covalent bond between B and L.
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X′ is the same or different and denotes a paramagnetic tripodal chelate consisting of a tripodal chelator X and a paramagnetic metal ion M; and
r r is the same or different and denotes the integer 2, 3 or 4; and
n denotes an integer of 3 to 6.

Hence, the compounds of formula (II) are compounds of formula (I) wherein X is a paramagnetic tripodal chelate X′.

In said preferred embodiment, said paramagnetic tripodal chelate X′ consists of the tripodal chelator X and a paramagnetic metal ion M, said tripodal chelator X and paramagnetic metal ion M form a complex which is denoted a paramagnetic tripodal chelate.

In the following M is a paramagnetic metal ion selected from ions of transition and lanthanide metals, i.e. metals of atomic numbers 21 to 29, 42 to 44 or 57 to 71. Preferably M is a paramagnetic metal ion of Mn, Fe, La, Co, Ni, Eu, Gd, Dy, Tm and Yb, particularly preferred a paramagnetic metal ion of Mn, Fe, La, Eu, Gd and Dy. Most preferably, M is selected from Gd³⁺, Mn²⁺, Fe³⁺, La³⁺, Dy³⁺ and Eu³⁺ with Gd³⁺ being the most preferred paramagnetic metal ion M.

In the following the term “ . . . X/X′”, e.g. in L′-X/X′ or in the formulae, means that the statement made or the drawn formula is equally suitable for compounds or residues comprising the tripodal chelator X or the paramagnetic tripodal chelate X′.

The compounds of the present invention according to formulae (I) and (II) are of the dendrimer type. Dendrimers are a class of polymer molecules with a central core with multiple branching arms including branching moieties that reproduces with an individual multiplicity. In the compounds of formulae (I) and (II) each branching arm is terminally linked to a chelator or a paramagnetic chelate The number of chelators or chelates in the compound depends on the number of branching moieties added to the structure, the multiplicity of the branching moieties and the number of branching arms on the core A. Depending on the number of branching moieties, and assuming that all branching moieties have a multiplicity of two, a compound with four branching arms will comprise of 8, 16, 32 or 64 chelators or chelates. A compound with three branching arms, each with branching moieties with a multiplicity of two, will comprise of 6, 12, 24 or 48 chelators or chelates. A compound of formula (II) with 8 chelates is shown in Compound A. The compound comprises a core A with four branching arms (n is 4), each arm comprising one branching moiety with an individual multiplicity of 2 (r is 2). Hence the compound has the formula A-(B-L-R-(L′-X′)₂)₄. Compound B shows a compound with one additional branching moiety on each of the two arms resulting from the first branching moiety. This compound has 16 chelates and the Compound A-(B-L-R-(L-R-(L′-X′)₂)₂)₄.

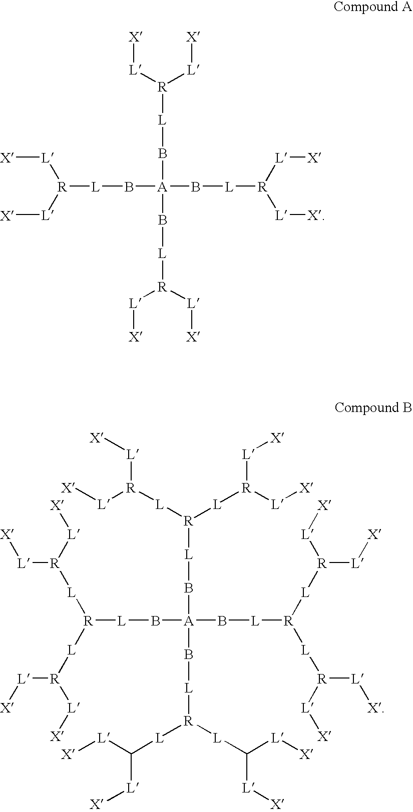

In the following the term “dendrimeric structure” denotes the compound of formula (I) or (II) without the linker moiety L′ and the chelator X or chelate X′.

The core A of the compounds of formula (I) and (II) preferably is a non-polymeric core. In another preferred embodiment, A is a cyclic core or a carbon atom having attached thereto 3 or 6 moieties B, wherein, when 3 moieties B are attached to said carbon atom, the forth valence may be hydrogen or a group selected from amino, hydroxyl, C₁-C₃-alkyl or halogen.

In one embodiment A is preferably a saturated or non-saturated, aromatic or aliphatic ring comprising at least 3 carbon atoms and optionally one or more heteroatoms N, S or O, said ring being optionally substituted with one or more of the following substituents: C₁-C₃-alkyl, optionally substituted with hydroxyl or amino groups, amino or hydroxyl groups or halogen, provided that there are n attachment points left for moieties B. Preferably, A is an aliphatic saturated or non-saturated 3- to 10-membered ring like cyclopropane, cyclobutane, cycloheptan or cyclohexane, which optionally comprises one or more heteroatoms N, S or O and which is optionally substituted with one or more substituents C₁-C₃-alkyl, optionally substituted with hydroxyl or amino groups, amino or hydroxyl groups or halogen, provided that there are 3 to 6 attachment points left for pendant moieties B. Alternatively, A is an aliphatic 3- to 10-membered ring optionally comprising one or more heteroatoms N, S, or O wherein one or more of the ring carbon atoms are carbonyl groups.

In another preferred embodiment, A is an aromatic single or fused 5- to 10-membered ring optionally comprising one or more heteroatoms N, S or O. Examples for such rings are for instance benzene or naphthalene. The aforementioned rings are optionally substituted with one or more substituents C₁-C₃-alkyl, optionally substituted with hydroxyl or amino groups, amino or hydroxyl groups or halogen, provided that there are at least 3 attachment points left for pendant moieties B.

In another preferred embodiment the core A is an ethyl group. The ethyl group may have attached thereto a maximum of 6 moieties B, wherein, when less than 6 moieties B are attached to said carbon atoms, the remaining valence(s) are hydrogen or a group selected from amino, hydroxyl, C₁-C₃-alkyl or halogen.

Preferred examples of core A are:

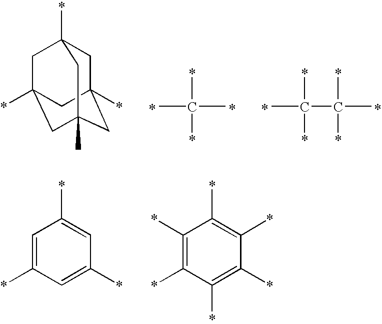

wherein,
* stands for the possible attachment points to B

In the compounds of formulae (I) and (II), B is the same or different and denotes a moiety that constitutes an obstacle for the rotation of the covalent bonds between B and L. This may be achieved by choosing a moiety B whose rotation is hindered by interaction with L, preferably sterical interaction.

Such sterical interaction occurs if B is a bulky moiety like an at least 5-membered carbocyclic or heterocyclic ring or a bicyclic or polycyclic ring. Such sterical interaction may further be promoted by using a bulky moiety B, e.g. the aforementioned bulky moieties which is substituted with C₁-C₃-alkyl, e.g. methyl, ethyl, n-propyl or isopropyl. Such bulky moieties B hinder the rotation of the B moiety due to interaction with L.

In a preferred embodiment B is selected from a residue of an optionally substituted aromatic or non-aromatic 5- to 7-membered carbocyclic or heterocyclic ring like pyridinyl, phenyl, substituted phenyl like benzyl, ethylbenzyl or cyclohexyl. In another preferred embodiment B is selected from a residue of an optionally substituted bicyclical or polycyclic ring like naphthyl or benzimidazolyl. Optional substituents are C₁-C₈-alkyl, hydroxyl, amino or mercapto groups or C₁-C₈-alkyl containing one or more hydroxyl or amino groups like CH₂OH, C₂H₄OH, CH₂NH₂ and/or an oxo-group like CH₂OCH3 or OC₂H₄OH.

The term “residues of . . . ” in the previous paragraph is chosen since B is attached to A and L. Thus, B is to be seen as a residue.

In a particularly preferred embodiment B is a residue of a 6-membered aromatic ring, preferably a benzene residue.

Preferred examples of B are:

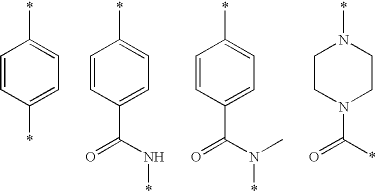

wherein,
* stands for the possible attachment points to A and L

In one embodiment the B moieties can be interconnected by covalent bonds.

Further, compounds of formula (I) and (II) are rigid compounds since the linker moiety L and the branching moieties R exert a rotation restriction.

L denotes a linker moiety that renders the compounds of formulae (I) and (II) compact and rigid. L is a covalent bond or can be chosen from the group:

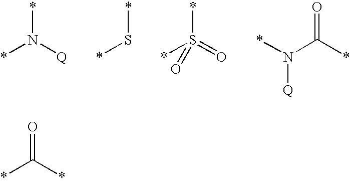

wherein
Q stands for H, C₁-C₈-alkyl, optionally substituted with one or more hydroxyl or amino groups; and
* stands for the possible attachment points to B and R

Preferably L is one of:

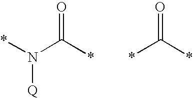

wherein Q and * have the meaning as described above.

Preferably, Q stands for H, C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

R denotes a branching moiety that reproduces with an individual multiplicity of r wherein r is 2, 3 or 4, with 2 being most preferred. The branching moiety exerts a rotation restriction and hence renders the branching arm rigid. This may be achieved by choosing a moiety R whose rotation is hindered by interaction with L and/or L′, preferably sterical interaction.

Preferred branching moieties are:

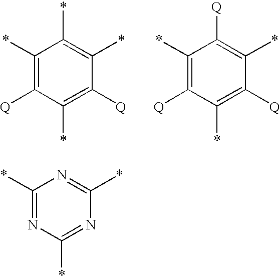

wherein,
Q is the same or different and has the meaning as described above; and
* stands for the possible attachment points to L, L′ and X/X′.

Preferably all Q are the same and Q is either H or CH₃.

Preferably all R are the same.

In compounds of formula (I) and (II), L′ may be present or not. If L′ is not present, R is directly linked to X (compounds of formula (I)) or X′ (compounds of formula (II)) via a covalent bond. If L′ is present, each L′ is the same or different and denotes a linker moiety, i.e. a moiety that is able to link R and X/X′.

Preferred examples of L′ are:

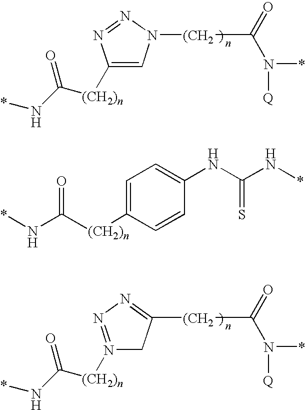

wherein
n is 0 to 6;
Q has the meaning as described above; and
* stands for the possible attachment points to X/X′ and R

The tripodal chelators X and paramagnetic tripodal chelates X′ hereinafter described in the present specification may exist in either solvated or unsolvated forms and both are encompassed within the scope of the present invention. The present invention also encompasses all solid forms of the compounds, including amorphous and all crystalline forms.

Certain X and X′ may exist in different isomeric forms and the present invention is intended to encompass all isomers including enantiomers, diastereoisomers and geometrical isomers as well as racemates.

Referring to the chelators X and chelates X′ in the present specification, the term “alkyl” by itself or as part of another substituent refers to a fully saturated straight or branched hydrocarbon chain group having the number of carbon atoms designated. Thus, C₁-C₆-alkyl means a fully saturated straight or branched hydrocarbon chain group having 1 to 6 carbon atoms and examples of C₁-C₆-alkyl are methyl, ethyl, n-propyl, isopropyl, n-butyl, isobutyl, t-butyl, n-pentyl, iso-pentyl and n-hexyl.

In a first preferred embodiment of the present invention the tripodal chelator X is one of the general formula (III) or (IV):

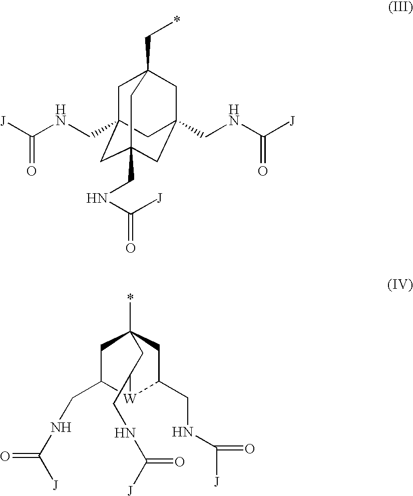

wherein
J is a chelator moiety consisting of a 6-membered aromatic or partially saturated ring system containing up to three heteroatoms selected from nitrogen and oxygen and having a hydroxyl group as a first substituent bound to a first atom in said ring system, and a hydroxyl group or an oxygen atom doubly bound to a second atom in said ring system wherein said first and second atom are adjacent atoms and wherein said first and second substituents are in ring positions such that J is capable of forming a complex with a paramagnetic metal ion; and wherein J is optionally substituted by up to three additional substituents, R¹, where each R¹ is independently a hydrophilic group which renders the compounds of formulae (III) and (IV) soluble in aqueous solutions;
* stands for the attachment point to L′ or R; and
W and the bonds represented as dotted lines are present or absent and when present, W is N.

In second preferred embodiment of the present invention the tripodal chelate X′ is of the general formula (V) or (VI), comprising the chelator X of formula (III) or (IV) and a paramagnetic metal ion M:

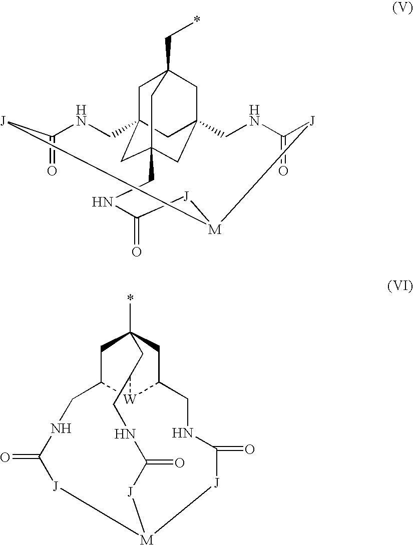

wherein
W, J, M and * are as defined above.

Examples of preferred chelators and chelates of formulae (IV) and (VI) are shown below as general formulae (IVa), (IVb), (VIa) and (VIb):

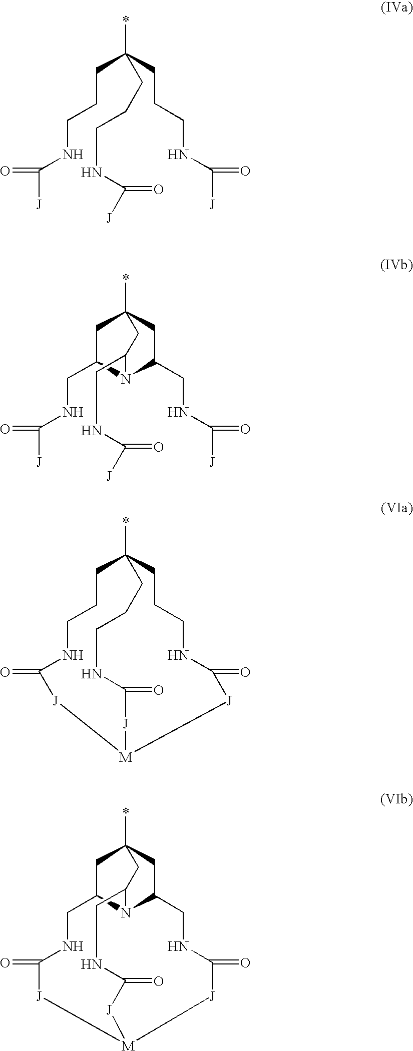

wherein J, M and * are as defined above.

Compounds of formulae (III), (IV), (V) and (VI) comprise a chelator moiety, i.e. group J. Preferred groups J include groups derived from hydroxypyrones, dihydroxypyridines, hydroxypyrimidones, hydroxypyridones hydroxypyridinones and dihydroxyphenols, any of which may be substituted as described above.

Groups J derived from hydroxypyridones and hydroxypyrimidinones are disclosed in US 2003/0095922.

Groups J derived from hydroxypyridinones which are capable of forming chelates with paramagnetic metal ions are also disclosed in U.S. Pat. No. 4,698,431, U.S. Pat. No. 4,666,927, U.S. Pat. No. 5,624,901 and our own earlier application number PCT/NO2008/000012.

Preferred groups J are of formula (VIIa) to (VIIg)

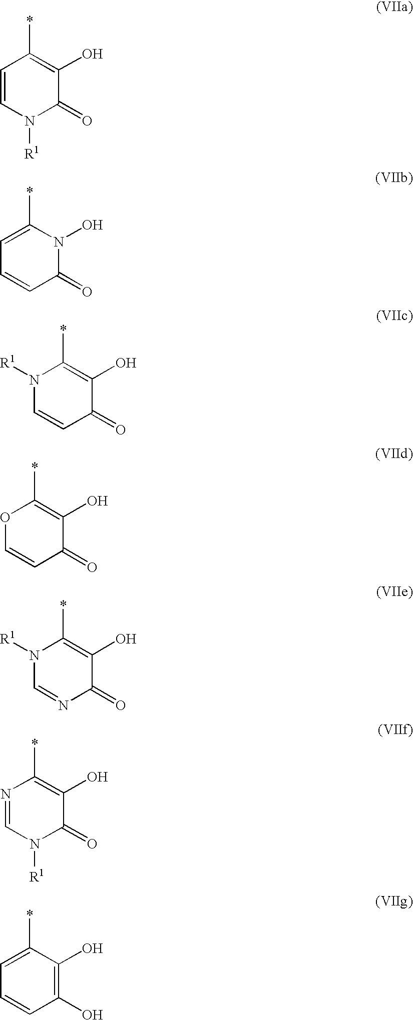

wherein R¹ is as defined in formulae (III), (IV), (V) and (VI) and * indicates the point of attachment of the group J to the remainder of the compound of formulae (III), (IV), (V) and (VI).

In compounds of formulae (V) and (VI) the chelator moieties J form a complex, i.e. paramagnetic chelate with a paramagnetic metal ion M where M is as defined above.

The chelator moieties J in compounds of formulae (III), (IV), (V) and (VI) may be substituted by up to three additional substituents, R¹, where each R¹ is independently a hydrophilic group which renders the compounds of formulae (V) and (VI) soluble in aqueous solutions.

Preferred hydrophilic groups R¹ are groups comprising ester groups, amide groups or amino groups which are optionally further substituted by one or more straight chain or branched C₁-C₁₀-alkyl groups, preferably C₁-C₅-alkyl groups where said alkyl groups also may have one or more CH₂- or CH-moieties replaced by oxygen or nitrogen atoms. The aforementioned preferred hydrophilic groups R¹ may further contain one or more groups selected from hydroxy, amino, oxo, carboxy, amide group, ester group, oxo-substituted sulphur and oxo-substituted phosphorus atoms. The aforementioned straight chain or branched C₁-C₁₀-alkyl groups, preferably C₁-C₅-alkyl groups, preferably contain 1 to 6 hydroxyl groups and more preferably 1 to 3 hydroxyl groups.

Particularly preferred hydrophilic groups R¹ according to the embodiment described above are the following groups R¹ which are attached to a carbon atom in the chelator moiety J and wherein said chelator moiety J is substituted by only one of said following groups R¹. * indicates the point of attachment of the group R¹ to J:

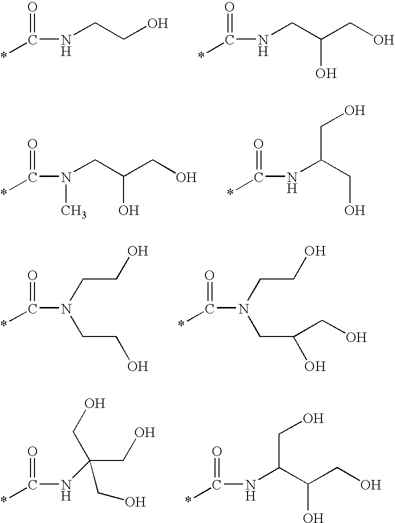

Further preferred hydrophilic groups R¹ are preferably attached to heteroatoms in the chelator moiety J, more preferably attached to nitrogen atoms in the chelator moiety J and such hydrophilic groups R¹ are straight chain or branched C₁-C₁₀-alkyl groups, preferably C₁-C₅-alkyl groups which are substituted by 1 to 6 hydroxyl groups and more preferably by 2 to 5 hydroxyl groups and/or which are substituted by one or more alkyloxy groups, preferably C₁-C₃-alkyloxy groups like methyloxy, ethyloxy and propyloxy groups.

Particularly preferred hydrophilic groups R¹ according to the embodiment described above are the following and * indicates the point of attachment of the group R¹ to J:

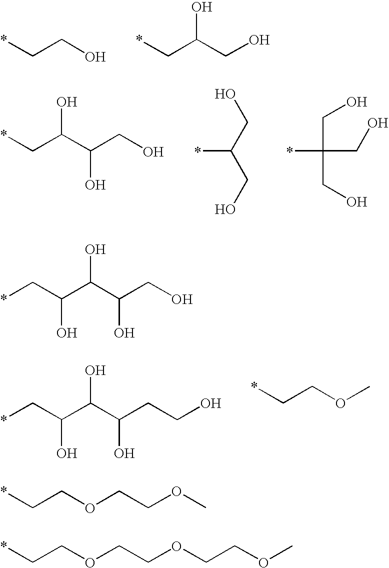

Further preferred hydrophilic groups R¹ are preferably attached to heteroatoms in the chelator moiety J more preferably attached to nitrogen atoms in the chelator moiety J and such hydrophilic groups R¹ are groups that comprise up to 3 ethylene oxide units.

Particularly preferred hydrophilic groups R¹ according to the embodiment described above are the following and * indicates the point of attachment of the group R¹ to J:

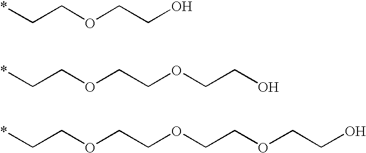

If linker moiety L′ is present chelators of formula (III) or (IV) or chelates of formula (V) or (VI) are linked via L′ to R in formula (I) or (II) respectively.

The term “linked via L′” means that derivatives of X/X′ and R comprising functional groups that are precursors to L′, are linked by reacting said functional groups thereby forming the linker L′. It is apparent for the skilled person that certain functional groups as defined below, i.e. a NH₂-group, are functional groups which can be converted to numerous other functional groups by methods known in the art. Thus the invention also includes embodiments wherein the functional group of either or both derivatives of X/X′ or R as defined is first converted into another functional group before resulting in the precursor to L′. The chelators and chelates of formula (III), (IV), (V) or (VI) derivatized with a precursor to L′ are reacted with derivatives of R that also are derivatized with a compatible precursor to L′. It is apparent for the skilled person which pair of functional groups that are compatible in that sense that they can be seen as precursors to L′.

Preferred examples of precursors to L′ to be attached either to X/X′ or R are:

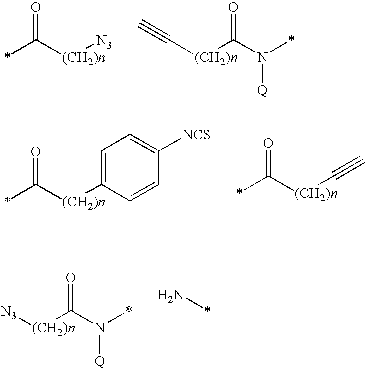

wherein
Q has the meaning as described above; and
stands for the attachment point to X/X′ and R.

In the following preferred precursors to L′ are denoted NHC(═O)R² wherein R² is as follows and * denotes the attachment point of R² to the carbon atom of group NHC(═O)R²:

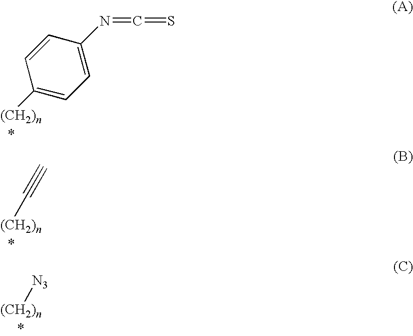

By using chelators or chelates of formula (III), (IV), (V) or (VI) derivatized with the aforementioned precursors to L′, it is possible to use “click chemistry” (e.g. described by M. Malkoch et al., Macromolecules 38(9), 2005, 3663-3678 or P. Wu et al., Chem. Commun. 46, 2005, 5775-5777). Click chemistry allows linking single or preferably multiple chelators or chelates of formula (III), (IV), (V) or (VI) to R by reacting the corresponding precursors to L′ in a very high yielding reaction. Further, the linking reaction can be carried out in conditions that dissolve the reactants such as aqueous conditions.

Chelators or chelates of formula (III), (IV), (V) or (VI) derivatized with a precursor to L′ wherein the precursor is NHC(═O)R² and R² is (A), i.e. (CH₂)ₙ—(C₆H₄)—NCS, can be reacted with R derivatized with a precursor to L′ wherein the precursor is comprising amino groups —NH₂ under formation of thiourea bonds (—NH—C(═S)—NH—).

Chelators or chelates of formula (III), (IV), (V) or (VI) derivatized with a precursor to L′ wherein the precursor is NHC(═O)R² and R² is (B), i.e. (CH₂)ₘ—C≡CH, can be reacted with R derivatized with a precursor to L′ wherein the precursor is comprising azido groups —N₃ under formation of 1,2,3 triazole rings.

Chelators or chelates of formula (III), (IV), (V) or (VI) derivatized with a precursor to L′ wherein the precursor is NHC(═O)R² and R² is (C), i.e. (CH₂)ₘ—N₃, can be reacted with R derivatized with a precursor to L′ wherein the precursor is comprising ethynyl groups-C≡CH under formation of 1,2,3 triazole rings.

For the synthesis of chelators of formula (IV) derivatized with a precursor to L′ compounds of formula (VIIIa) and (VIIIb) are useful starting materials:

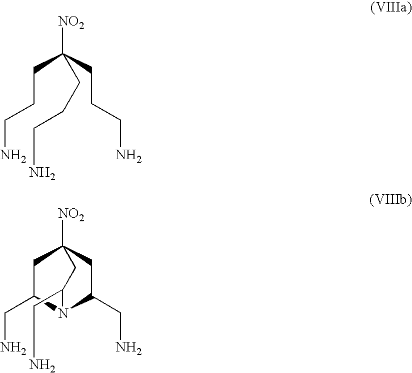

The compound of formula (VIIIa) can be prepared from tris-(2-cyanoethyl)nitromethane, a commercially available compound, which is reduced with BH₃/THF complex as described by S. Lebreton et al., Tetrahedron 59 (2003), 3945-3953.

The compound of formula (VIIIb) can be prepared as shown in reaction scheme 1:

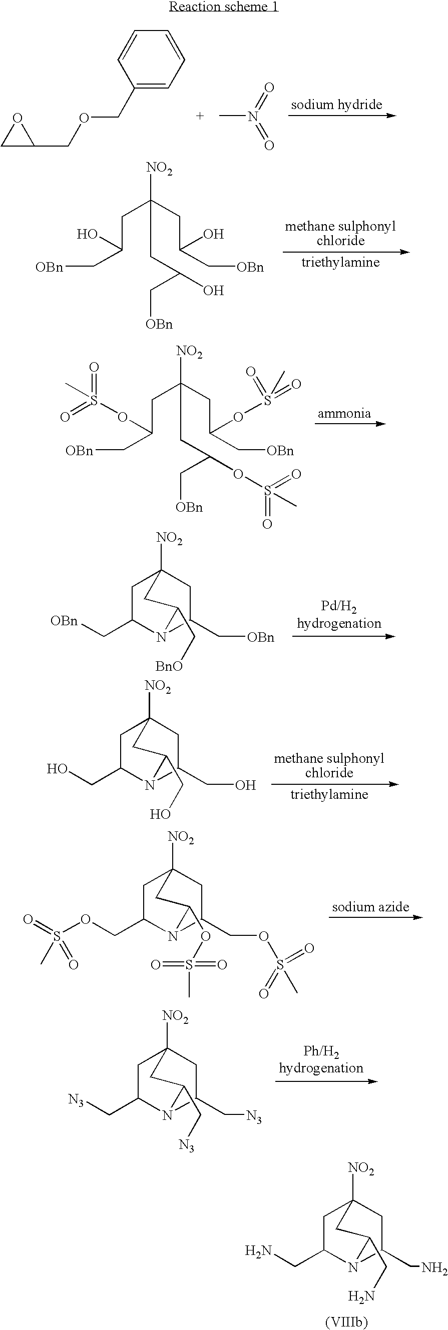

Excess benzyl glycidyl ether is reacted with nitromethane with base such as potassium tert.-butoxide in a solvent such as THF. The resulting triol is treated with methane sulphonyl chloride and a base such as triethylamine or pyridine in a solvent such as dichloromethane. The trimethane sulphonate is then reacted with ammonia in a solvent such as THF. The benzyl protecting groups are then removed by hydrogenation with palladium on charcoal to give the nitro triol. The triol functions are then converted to leaving groups by reaction with an activating group such as methane sulphonyl chloride in a solvent such as THF or dichloromethane. The leaving groups are then reacted with sodium azide to displace them and give the triazide. The azido groups can then be hydrogenated over palladium on charcoal to give the compound of formula (VIIIb) in a solvent such as methanol.

Compounds of formula (VIIIa) and (VIIIb) are then reacted with a compound of formula (IX) comprising the chelating moiety J in a protected form and a leaving group:

J^{Z}-C(═O)G (IX)

wherein
J^{Z} is J as defined before wherein the hydroxyl groups which are bound to J are protected; and
G is a leaving group, preferably a halide, a mixed anhydride, an activated ester such as O-succinimide or an activated amide such as imidazolide.

The product obtained may then be reduced and deprotected to obtain chelators of formula (IV) derivatized with NH₂.

Thus, a chelator of formula (IV) derivatized with NH₂ can be produced by
a) reacting a compound of formula (VIIIa) or (VIIIb)

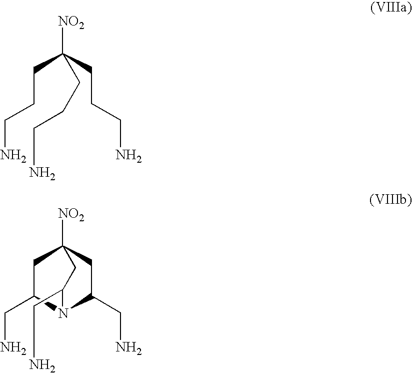

with a compound of formula (IX)

J^{Z}-C(═O)G (IX)
wherein
J^{Z} is J as defined earlier and wherein the hydroxyl groups which are bound to J are protected; and
G is a leaving group;
b) reducing the nitro group to obtain an amino group; and
c) removing the hydroxyl protecting groups of J^{Z}.

The hydroxyl group(s) present in J, i.e. attached to the ring system need to be protected. Suitable protecting groups for hydroxyl groups are well known in the art and are for instance described in “Protecting Groups in Organic Synthesis”, Theodora W. Greene and Peter G. M. Wuts, published by John Wiley & Sons Inc. Examples of suitable groups protecting groups for hydroxyl groups include tert.-butyl groups or benzyl, with benzyl being preferred.

If J contains one or more substituents R¹, hydroxyl groups present in R¹ may or may not be protected. If R¹ comprise other reactive groups than the aforementioned hydroxyl groups, e.g. such as amine groups, such groups need to be protected as well. Again suitable protecting groups are well known in the art.

The reaction of compounds of formula (VIIIa) or (VIIIb) with compounds of formula (IX) is preferably conducted in organic solvent(s) such as dichloromethane or tetrahydrofuran (THF) under anhydrous conditions but for some reagents, an aqueous solution may be used. The reaction of compounds of formulae (VIIIa) or (VIIIb) with compounds of formula (IX) gives compounds of formulae (Xa) or (Xb), respectively.

The reaction is illustrated in reaction scheme 2:

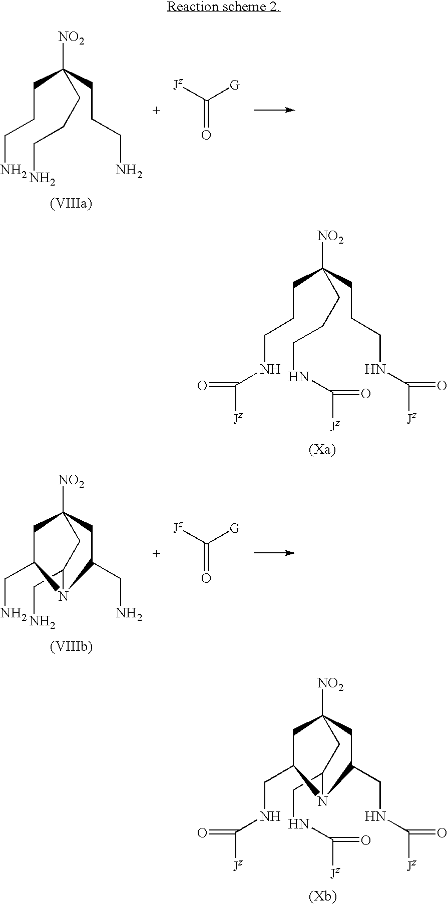

Compounds of formula (IX) are also known and may be prepared by known methods. For example, compounds of formula (IX) in which J is a group of formula (VIIa) are designated compounds of formula (IXa):

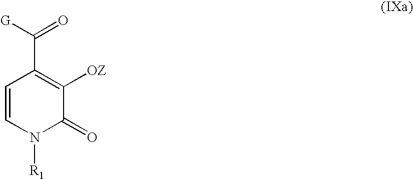

wherein R¹ and G are as defined above and Z is a protecting group for OH as described above.

Compounds (IXa) may be prepared by reacting compounds of formula (XI) which are well known in the art:

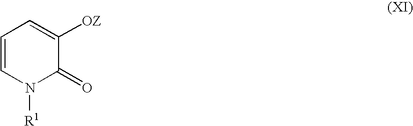

wherein R and Z are as defined above, with carbon dioxide in the presence of a base. A suitable method for this reaction is set out in U.S. Pat. No. 5,624,901.

Other compounds of formula (IX) which have a different J group, for example an J group of formula (VIIb), (VIIe), (VIIf) and (VIIg) can be prepared by methods similar to those above or methods known to those skilled in the art and set out in, for example US-A-2003/0095922, Z. Liu et al., Bioorg. Med. Chem. 9 (2001), 563-573, S. Piyamongkol et al., Tetrahedron Letters 46 (2005), 1333-1336, V. Pierre et al., J. Am. Chem. Soc. 2006, 128, 5344-5345, J. Xu et al., J. Am. Chem. Soc. 1995, 117, 7245-7246, D. Doble et al., J. Am. Chem. Soc. 2001, 123, 10758-10759, M. Allen et al., J. Am. Chem. Soc. 2006, 128, 6534-6535, M. Seitz et al., Inorg. Chem. 2007, 46, 351-353, K. Clarke Jurchen et al., Inorg. Chem. 2006, 45, 1078-1090, B. O'Sullivan et al., Inorg. Chem. 2003, 42, 2577-2583, D. Doble et al., Inorg. Chem. 2003, 42, 4930-4937, S. Dhungana et al., Inorg. Chem. 2001, 40, 7079-7086, A. Johnson et al., Inorg. Chem. 2000, 39, 2652-2660, S. Cohen et al., Inorg. Chem. 2000, 39, 4339-4346.

Compounds of formula (IXc) which have a J group of formula (VIIc):

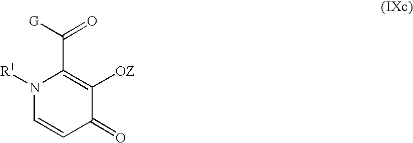

are preferably produced as illustrated in reaction scheme 3, wherein G′ denotes a precursor of G:

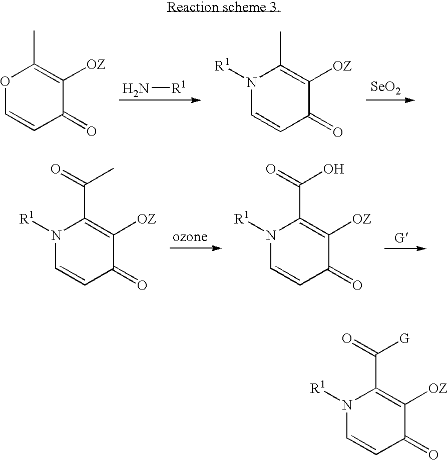

By deprotecting compounds of formula (Xa) and (Xb), chelates of formula (IV) derivatized with NO₂ are obtained. By reducing compounds of formula (Xa) and (Xb) and deprotecting the reaction product from that reduction reaction, chelators of formula (IV) derivatized with NH₂ are obtained.

Hence, compounds of formula (Xa) and (Xb) are suitable starting compounds for the synthesis of chelators of formula (IV) derivatized with NH₂ or NHC(═O)R², wherein R² is (CH₂)ₙ—(C₆H₄)—NCS, (CH₂)ₙ—C≡CH or (CH₂)ₙ—N₃ wherein n is 0 to 6.

Thus, there is provided a method for producing a chelator of formula (IV) derivatized with a precursor to L′ wherein the precursor is NHC(═O)R², wherein R² is (CH₂)ₙ—C≡CH or (CH₂)n-N₃ wherein n is 0 to 6 by
a) reacting a compound of formula (VIIIa) or (VIIIb)

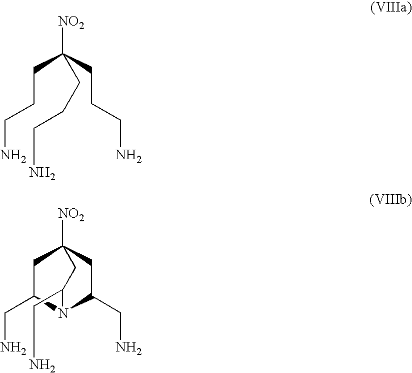

with a compound of formula (IX)

J^{Z}-C(═O)G (IX)
wherein
J^{Z} is J as defined earlier and wherein the hydroxyl groups which are bound to J are protected; and
G is a leaving group;
b) reducing the nitro group to obtain an amino group;
c) reacting the product obtained with a compound of formula (XII)

G-C(═O)R² (XII)
wherein Y and R² are as defined above; and
d) removing the hydroxyl protecting groups of J^{Z}.

Thus, in another aspect the invention provides a method for producing a chelator of formula (IV) derivatized with a precursor to L′ wherein the functional group is NHC(═O)R² and R² is (CH₂)ₙ—(C₆H₄)—NCS wherein n is 0 to 6 by
a) reacting a compound of formula (VIIIa) or (VIIIb)

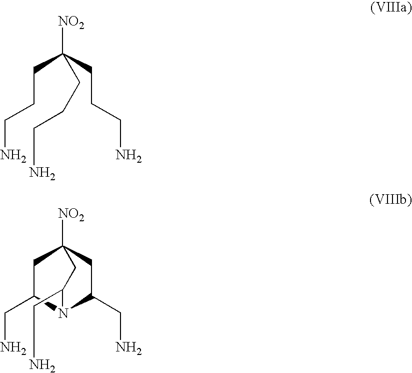

with a compound of formula (IX)

J^{Z}-C(═O)G (IX)
wherein
J^{Z} is J as defined earlier and wherein the hydroxyl groups which are bound to J are protected; and
G is a leaving group;
b) reducing the nitro group to obtain an amino group;
c) reacting the product obtained with a compound of formula (XII_{A*})

G-C(═O)—(CH₂)ₙ—(C₆H₄)—NO₂ (XII_{A*})
wherein G and n are as defined above;
d) reducing the nitro group to an amino group;
e) reacting the amino group with thiophosgene to give the isothiocyanate group —N═C═S; and
f) removing the hydroxyl protecting groups of J^{Z}.

Compounds of formula (XII_{A*}) may be prepared by for instance reacting a carboxylic acid of formula HOOC—(CH₂)ₙ—(C₄H₆)—NO₂ and N-hydroxysuccinimide in the presence of a coupling agent such as dicyclohexylcarbodiimide (DCC)

Compounds of formula (XII) wherein R² is (B) may be prepared by for instance reacting an ω-alkynoic acid HOOC—(CH₂)ₙ—C≡CH with N-hydroxysuccinimide in the presence of a coupling agent such as DCC.

Compounds of formula (XII) wherein R² is (C) may be prepared by for instance reacting an ω-azido carboxylic acid HOOC—(CH₂)ₙ—N₃ with N-hydroxysuccinimide in the presence of a coupling agent such as DCC.

Chelators of formula (IV) derivatized with NH₂ are readily obtained from compounds of formula (Xa) and (Xb) by reducing the nitro group present in these compounds by hydrogenation with a Rayney nickel catalyst in a solvent such as methanol. If chelators of formula (IV) derivatized with NH₂ are the desired end product, the protecting groups for hydroxyl groups in the chelator moiety J are removed by for instance hydrogenation with a palladium catalyst or acid cleavage of benzyl protection groups. It is also possible to reduce the nitro group and remove the protecting groups for hydroxyl groups simultaneously, e.g. by using a catalyst mixture of Rayney nickel and palladium.

If chelators of formula (IV) derivatized with NH₂ are used as starting compounds for the synthesis of chelators of formula (IV) derivatized with a precursor to L′ wherein the precursor is NHC(═O)R², wherein R² is (CH₂)ₙ—(C₆H₄)—NCS, (CH₂)ₙ—C≡CH or (CH₂)ₙ—N₃ wherein n is 0 to 6, the hydroxyl protecting groups in the chelator moiety J may or may not be present. If R² is (CH₂)ₙ—(C₆H₄)—NCS the hydroxyl protecting groups should not be present as the deprotection (hydrogenation) is poisoned by the presence of an isothiocyanate group —NCS. For all other cases, it is preferred that the hydroxyl protecting groups are present since it was observed that their presence gives better yields.

The reactions discussed above are illustrated in reaction scheme 4:

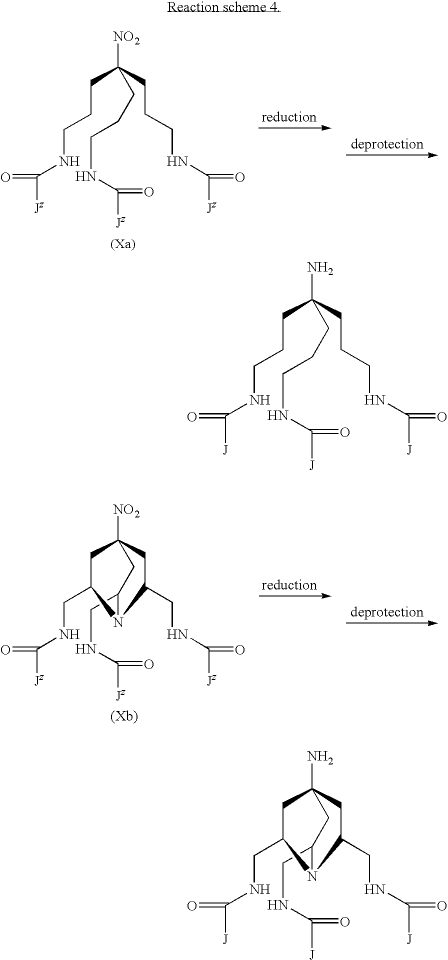

In the following, optionally protected chelators of formula (IV) derivatized with NH₂ are denoted compounds of formula (XIIIa) and (XIIIb)

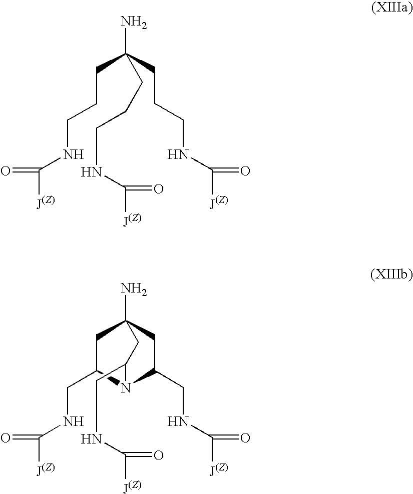

and J^{(Z)} indicates that the hydroxyl protecting group Z may or may not be present in the chelator J of said compounds.

Chelators of formula (IV) derivatized with a precursor to L′ wherein the precursor is NHC(═O)R² and R² is (B) or (C), i.e. (CH₂)ₙ—C≡CH or (CH₂)ₙ—N₃ can be prepared by reacting a compound of formula (XII)

G-C(═O)R² (XII)

wherein R² is (B) or (C) as defined above and G is a leaving group as defined above with compounds of formula (XIIIa) or (XIIIb) to result compounds of formula (XIVa) and (XIVb). This reaction is illustrated in reaction scheme 5:

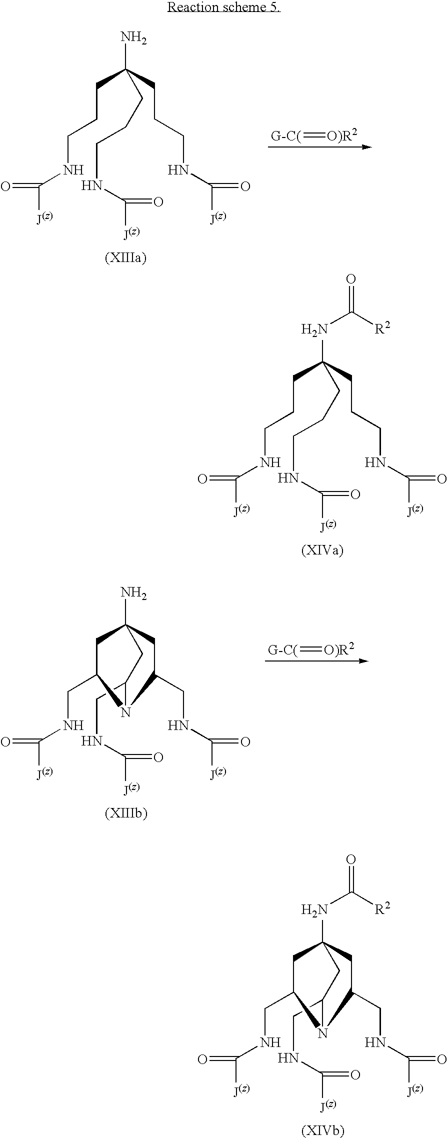

Chelators of formula (IV) derivatized with a precursor to L′ wherein the precursor is NHC(═O)R² and R² is (A), i.e. (CH₂)ₙ—(C₄H₆)—NCS can be prepared by
a) reacting a compound of formula (XII_{A*})

G-C(═O)—(CH₂)ₙ—(C₆H₄)—NO₂ (XII_{A*})
wherein n is defined as above and G is a leaving group as defined above with compounds of formula (XIIIa) or (XIIIb);
b) reducing the nitro group to an amino group; and
c) reacting the amino group with thiophosgene to give the isothiocyanate group —N═C—S.

Compounds of formula (XII_{A*}) may be prepared as described above.

To produce derivatives of chelates of formula (VI), derivatives of chelators of formula (IV) obtained by the synthetic routes discussed above are reacted with a chosen paramagnetic metal ion M, preferably in the form of its salt, e.g. nitrate, chloride, acetate and sulphate salts, in water as a solvent. Alternatively, an oxide of said chosen paramagnetic metal ion M may be used, e.g. Gd₂O₃, and a solution of the derivatives of the chelators of formula (IV) is then stirred with said oxide. This method is often preferred since it avoids the problem of free residual paramagnetic metal ions being present in the reaction product.

Thus the invention provides a method for producing derivatives of chelates of formula (VI) by reacting derivatives of chelators of formula (IV) with a paramagnetic metal ion, preferably in the form of its salt or in the form of its oxide.

Compounds of formula (XIVa) or (XIVb) wherein R² is (A) are readily reacted with the dendrimeric structure containing a precursor to L′ comprising amino groups. This reaction is shown in reaction scheme 6A:

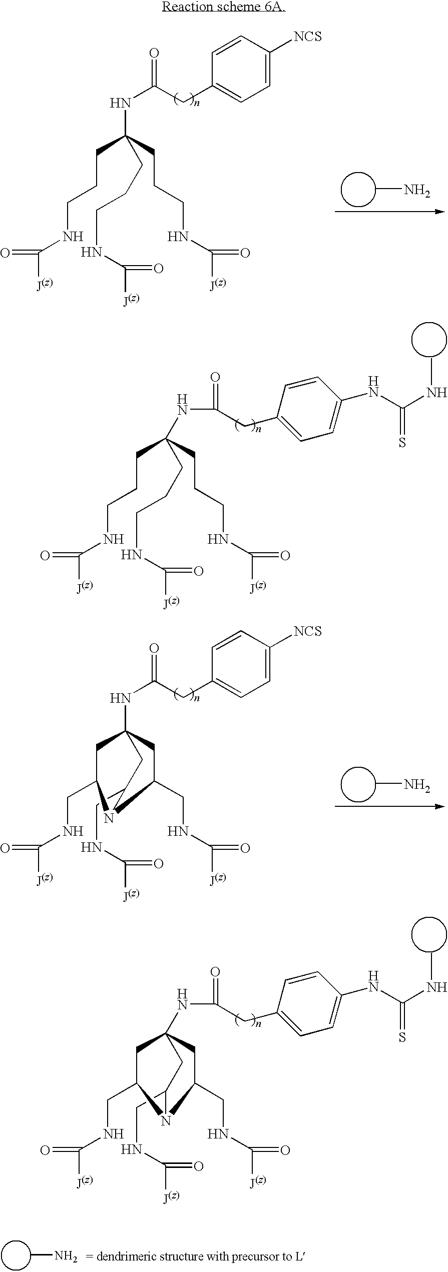

Compounds of formula (XIVa) or (XIVb) wherein R² is (B) are readily reacted with the dendrimeric structure containing a precursor to L′ comprising amino groups. This reaction is shown in reaction scheme 6B. In a preferred embodiment, this reaction is catalysed by a copper catalyst prepared by for instance, reacting copper sulphate with ascorbic acid.

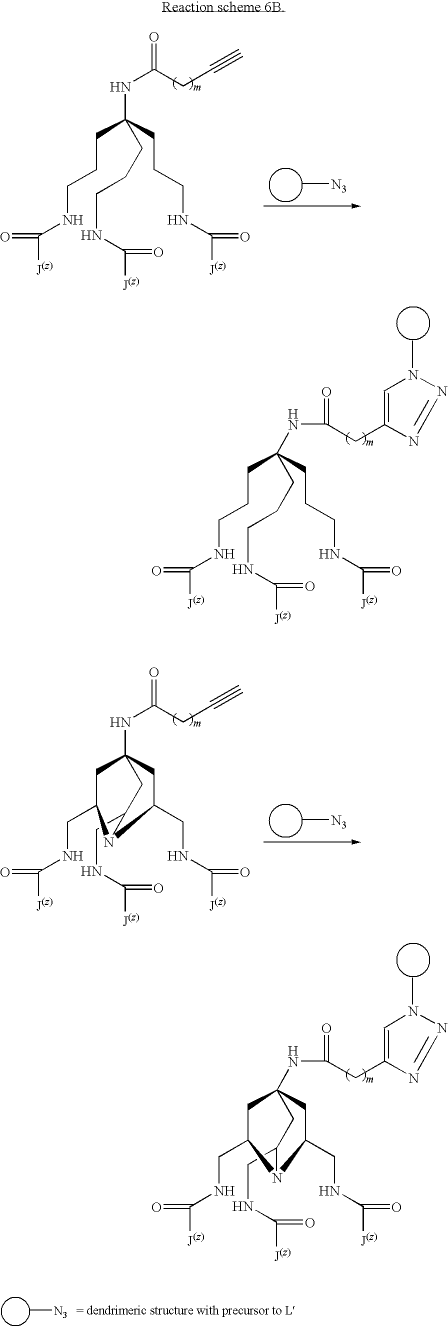

Compounds of formula (XIVa) or (XIVb) wherein R² is (C) are readily reacted with the dendrimeric structure containing a precursor to L′ comprising amino groups. This reaction is shown in reaction scheme 6C. In a preferred embodiment, this reaction is catalysed by a copper catalyst prepared by for instance, reacting copper sulphate with ascorbic acid.

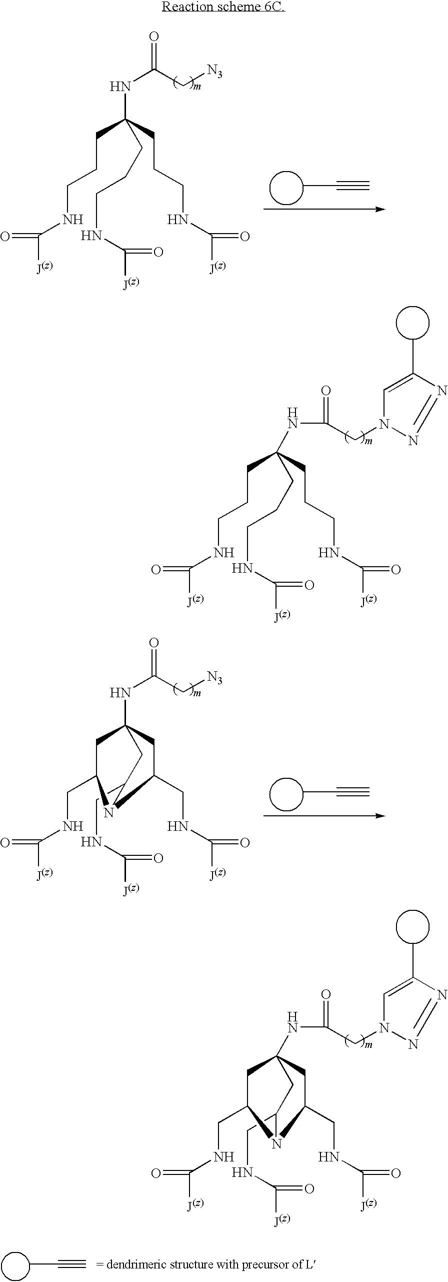

Chelators of formula (III) derivatized with a precursor to L′ may be prepared by using 1,3,5,7-tetrakis(aminomethyl)adamantane as a starting material. 1,3,5,7-tetrakis(amino-methyl)adamantane may be obtained as described by G. S. Lee et al., Org. Lett. Vol. 6, No. 11, 2004, 1705-1707. Briefly, adamantane is reacted with AlBr₃/Br₂ to tetrabromoadamantane whose subsequent photolysis with NaCN in DMSO results in tetracyanoadamantane. 1,3,5,7-tetrakis(aminomethyl)adamantane is then obtained by reduction of tetracyanoadamantane with monochloroborane and reaction with dry methanolic HCl.

In a subsequent step, a mono-protected 1,3,5,7-tetrakis(aminomethyl)adamantane is produced which is then coupled to a compound of formula (IX) comprising J which is protected and a leaving group:

J^{Z}-C(O)-G (IX)

Wherein J^{Z} and G are as described above.

The hydroxyl group(s) present in J, i.e. attached to the ring system need to be protected. Suitable protecting groups for hydroxyl groups are well known in the art and are for instance described in “Protecting Groups in Organic Synthesis”, Theodora W. Greene and Peter G. M. Wuts, published by John Wiley & Sons Inc. Examples of suitable groups protecting groups for hydroxyl groups include tert.-butyl groups or benzyl, with benzyl being preferred.

If J contains one or more substituents R¹, hydroxyl groups present in R¹ may or may not be protected. If R¹ comprise other reactive groups than the aforementioned hydroxyl groups, e.g. such as amine groups, such groups need to be protected as well. Again suitable protecting groups are well known in the art.

The reaction of mono-protected 1,3,5,7-tetrakis(aminomethyl)adamantane with compounds of formula (IX) is preferably conducted in organic solvent(s) such as dichloromethane or tetrahydrofuran (THF) under anhydrous conditions but for some reagents, an aqueous solution may be used.

The reaction is illustrated in reaction scheme 7:

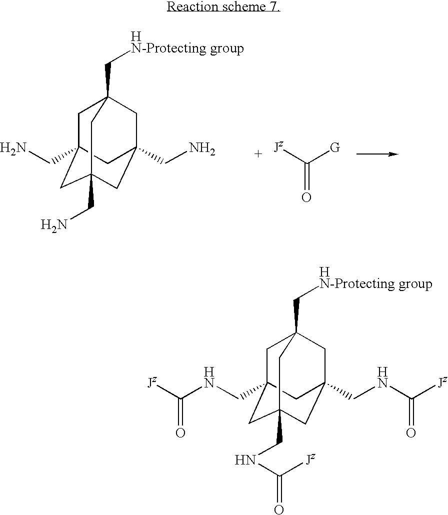

Suitable protecting groups for amines are known in the art and a mono-protected 1,3,5,7-tetrakis(aminomethyl)adamantane can be obtained by reacting 1 equivalent 1,3,5,7-tetrakis(aminomethyl)adamantane with ¼ equivalent of a precursor, e.g. an acyl chloride or anhydride, of the chosen protection group. A preferred precursor of such a protecting group is benzyl chloroformate or BOC anhydride (di-tert-butyl dicarbonate)

Compounds of formula (IX) may be prepared as described above

In a subsequent reaction the protecting groups Z and the amino protecting groups are removed by methods known in the art and chelators of formula (III) derivatized with NH₂ are obtained. Said subsequent reaction is illustrated in reaction scheme 8:

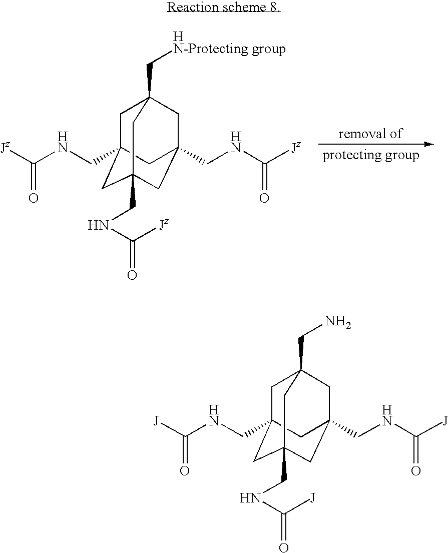

The removal of said protecting groups is done in a two-step procedure. In a first step, the amino protecting group is removed and the free amino group may be reacted with a suitable compound to give chelators of formula (III) derivatized with functional groups, e.g. to react said derivatives with R derivatized with a precursor to L′. In a second step, the protecting groups Z are removed.

Chelators of formula (III) derivatized with NH₂ are useful starting materials for the production of chelates of formula (V) derivatized with other precursors to L′ which are to be reacted with R derivatized with a precursor to L′. If chelators of formula (III) derivatized with NH₂ are to be used as such starting materials, the hydroxyl protecting groups of J^{Z} are preferably not removed.

Thus, in yet another aspect the invention provides a method for producing chelators of formula (III) derivatized with precursors to L′ wherein the precursor is NHC(═O)R² and R² is (CH₂)ₙ—C≡CH or (CH₂)ₙ—N₃ by
a) reacting a mono-protected 1,3,5,7-tetrakis(amino-methyl)adamantane with a compound of formula (IX)

J^{Z}-C(O)-G (IX)
wherein
J^{Z} is J as defined earlier and wherein the hydroxyl groups which are bound to J are protected; and
G is a leaving group;
b) removing the amino protecting group of said mono-protected 1,3,5,7-tetrakis(aminomethyl)adamantane;
c) reacting the product obtained with a compound of formula (XII); and

G-C(═O)R² (XII)
d) removing the hydroxyl protecting groups of J^{Z}.

In yet another aspect the invention provides a method for producing chelators of formula (III) derivatized with precursors to L′ wherein the precursor is NHC(═O)R² and R² is (CH₂)n—(C₆H₄)—NCS by

a) reacting a mono-protected 1,3,5,7-tetrakis(amino-methyl)adamantane with a compound of formula (IX)

J^{Z}-C(O)-G (IX)
wherein
J^{Z} is J as defined earlier and wherein the hydroxyl groups which are bound to J are protected; and
G is a leaving group;
   b) removing the amino protecting group of said mono-protected 1,3,5,7-tetrakis(aminomethyl)adamantane;
   c) reacting the product obtained with a compound of formula (XII_{A*})

G-C(═O)—(CH₂)ₙ—(C₆H₄)—NO₂ (XII_{A*})
wherein n and G are as defined above;
   d) reducing the nitro group to an amino group;
   e) reacting the amino group with thiophosgene to give the isothiocyanate group —NCS; and
   f) removing the hydroxyl protecting groups of J^{Z}.

Compounds of formula (XII_{A*}) may be prepared as described above.

To produce derivatives of chelates of formula (V) from derivatives of chelators of formula (III) the derivatives of chelators of formula (III) obtained in the reaction illustrated above are reacted with a chosen paramagnetic metal ion M, preferably in the form of its salt, e.g. nitrate, chloride, acetate and sulphate salts, in water as a solvent. Alternatively, an oxide of said chosen paramagnetic metal ion M may be used, e.g. Gd₂O₃, and a solution of the derivative of the chelate of formula (V) is then stirred with said oxide. This method is often preferred since it avoids the problem of free residual paramagnetic metal ions being present in the reaction product.

Chelators of formula (III) derivatized with NHC(═O)R² and R² is (B), i.e. (CH₂)ₙ—C≡CH or (C), i.e. (CH₂)ₙ—N₃ can be prepared by reacting a compound of formula (XII)

G-C(═O)R² (XII)

wherein R² is (B) or (C) as defined above and G is a leaving group as defined above with an optionally protected chelator of formula (III) derivatized with NH₂.

Compounds of formula (XII) may be prepared as described above.

Chelators of formula (III) derivatized with C(═O)R² and R² is (A), i.e. (CH₂)ₙ—(C₆H₄)—NCS can be prepared by
a) reacting a compound of formula (XII_{A*})

G-C(═O)—(CH₂)ₙ—(C₆H₄)—NO₂ (XII_{A*})

wherein
n is defined as above and
G is a leaving group as defined above
with an optionally protected chelator of formula (III) derivatized with NH₂,
b) reducing the nitro group to an amino group; and
c) reacting the amino group with thiophosgene to give the isothiocyanate group —N═C═S.

Compounds of formula (XII_{A*}) may be prepared as described above.

Compounds of formula (XII) and (XII_{A*}) can be reacted with derivatives of chelators of formula (III) as shown in reaction scheme 9:

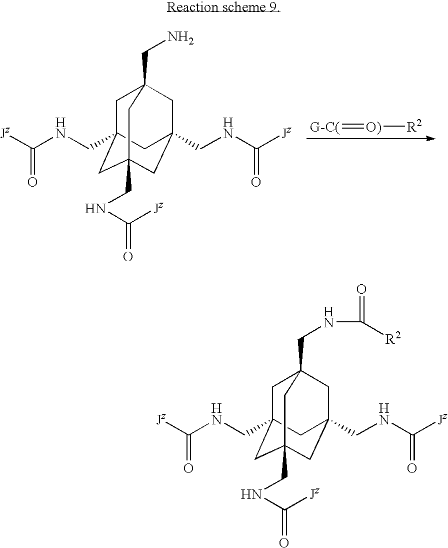

Chelators of formula (III) derivatized with NHC(═O)R² and R² is (A) are readily reacted with the dendrimeric structure containing a precursor to L′ comprising amino groups. This reaction is shown in reaction scheme 10A:

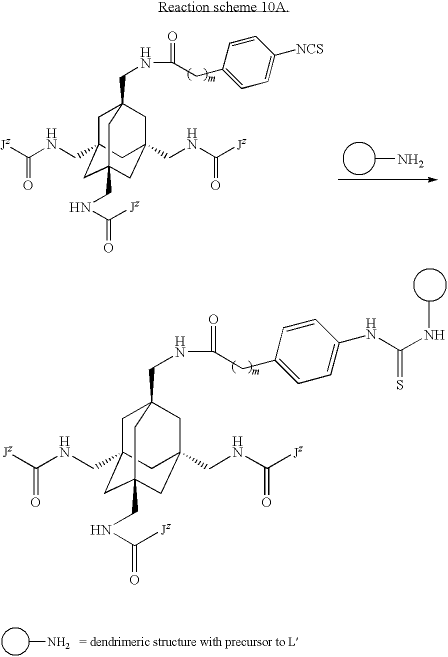

Chelators of formula (III) derivatized with NHC(═O)R² and R² is (B) are reacted with the dendrimeric structure containing a precursor to L′ comprising azido groups. This reaction is shown in reaction scheme 10B:

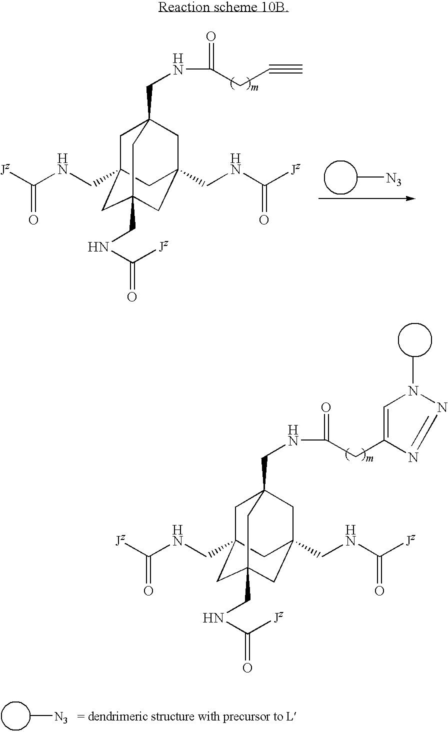

Chelators of formula (III) derivatized with NHC(═O)R² and R² is (C) are readily reacted with the dendrimeric structure containing a precursor to L′ comprising ethynyl groups. This reaction is shown in reaction scheme 10C:

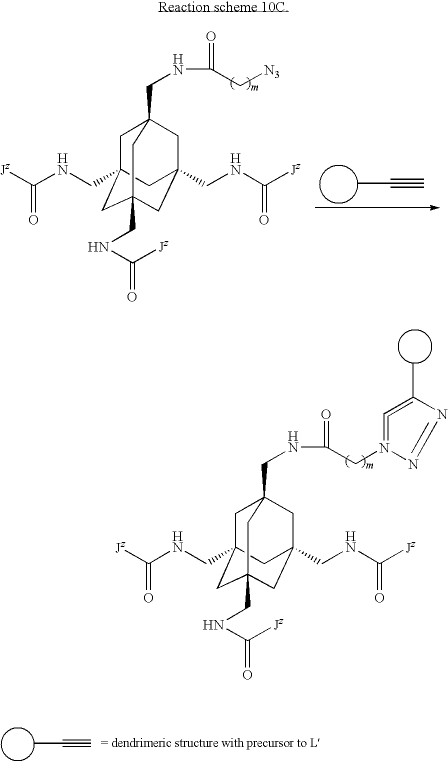

In compounds of formula (I) and (II) preferably all B are the same and/or all L are the same and/or all R are the same and/or all L′ are the same and/or all X/X′ are the same.

Preferred examples of compounds of formula (II) are:

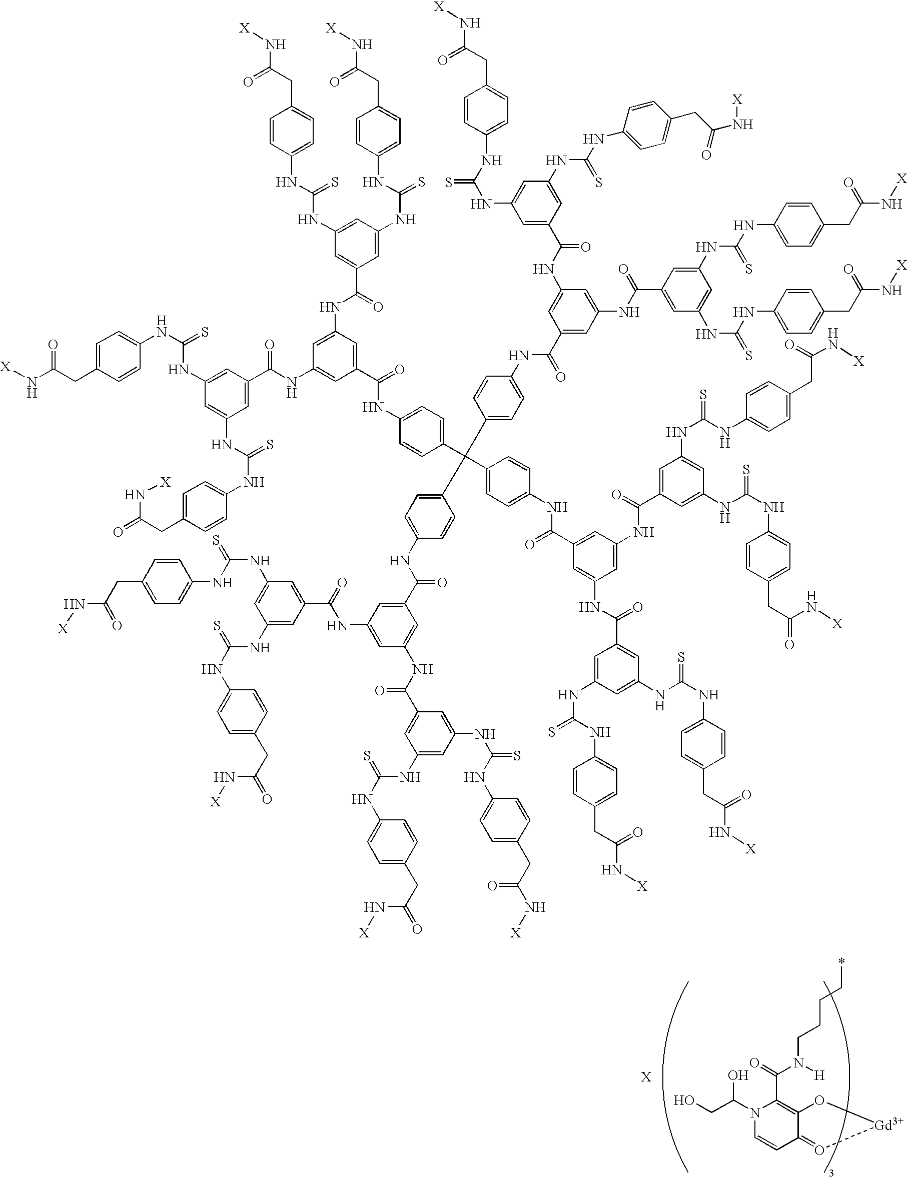

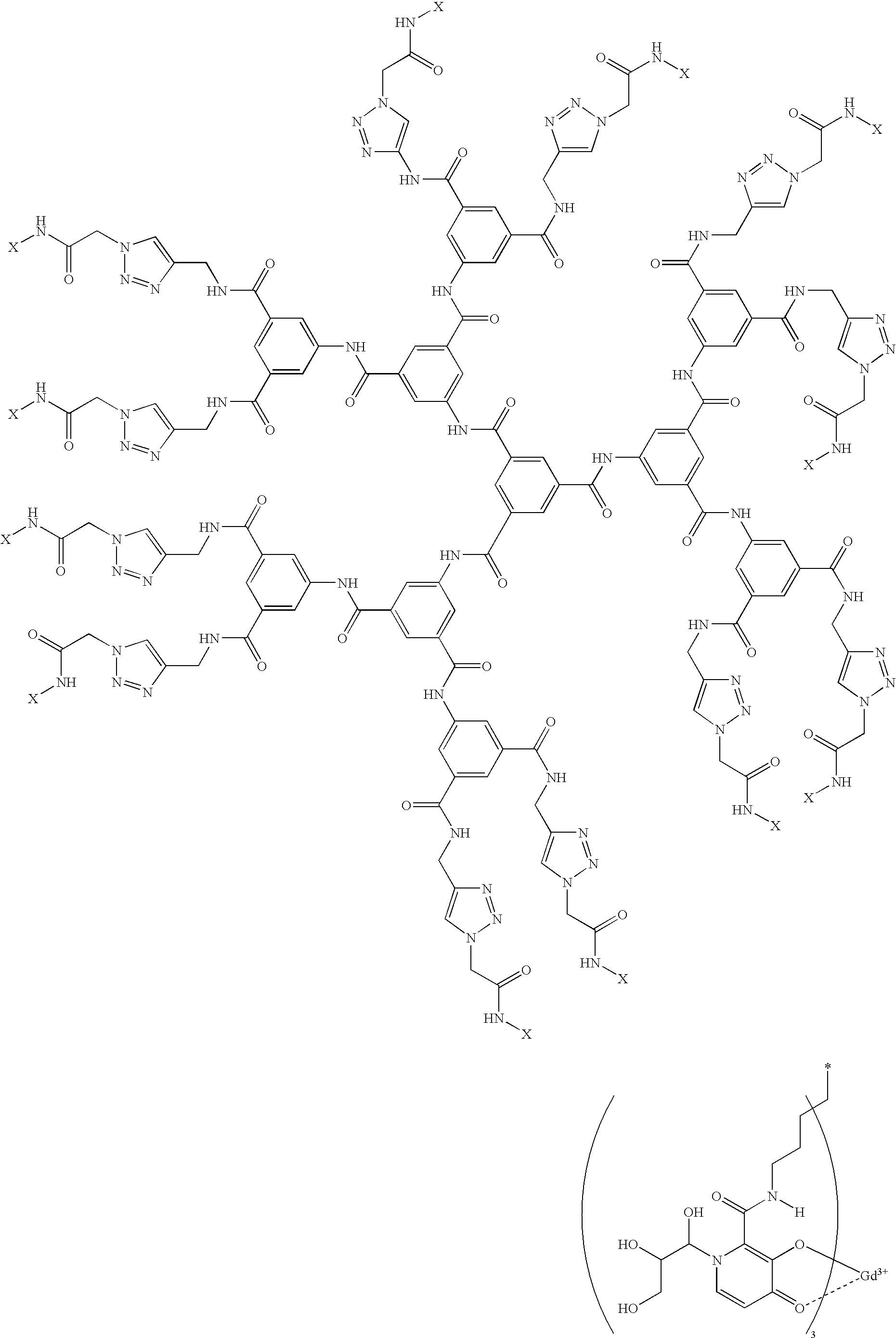

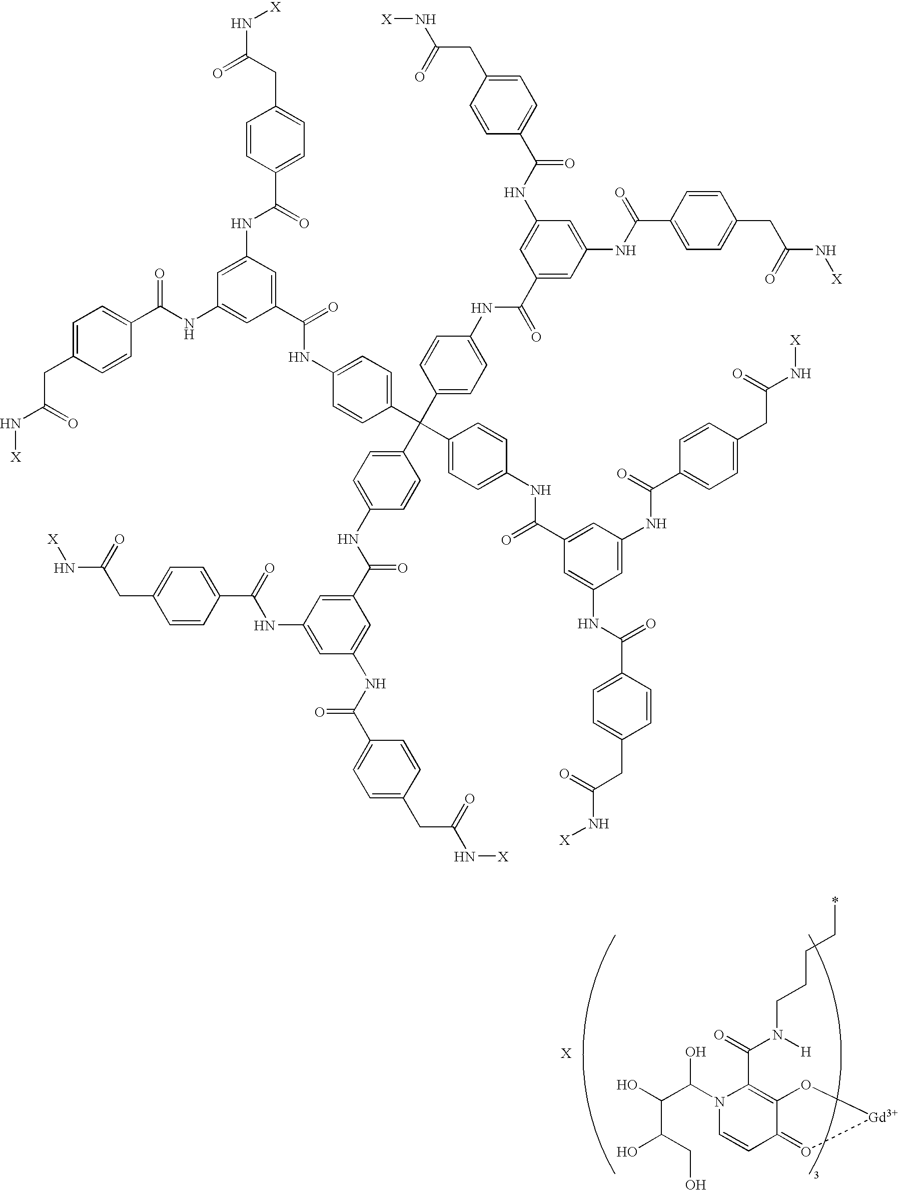

The compounds of formulae (I) and (II) can as discussed above be synthesized by several synthetic pathways known to the skilled artisan from commercially available starting materials by the following generalized process.

The compounds are preferably synthesized by a convergent approach where the individual building blocks are combined and attached to the core structure. In synthesizing a compound of formula A-(B-L-R-(L-R-(L-R-(L-R-(L′-X)ᵣ)ᵣ)ᵣ)ᵣ)ₙ, for example a precursor to the core A can be attached to a precursor to the moiety B. The attachment process is preferably based on an amide bond approach where one of the building blocks is equipped with an amine group and the other building block is equipped with an activated carboxylic acid. By reacting the two building blocks an amide bond will be formed. Alternatively an A-(B)ₙ block that is commercially available is provided.

The attached B moiety is also equipped with additional reactive groups albeit in a protected form. Examples of such are azide-, nitro-, amide- and carbamate-groups, which can be transformed in to an amine group, and ester-groups which can be transformed into an activated carboxylic acid group. The formed A-(B)ₙ building block can then be transformed into an activated form by modification of the latent protective groups, on the B moieties, into functional groups suitable for further attachment.

The A-(B)ₙ block can then be attached to a R group by forming a linker group L. The attachment process is preferably based on an amide bond approach where one of the building blocks is equipped with an amine group and the other building block is equipped with an activated carboxylic acid. By reacting the two building blocks a linker group L will be formed.

The A-(B-L-R)ₙ block can then either be linked to an additional generation of R groups, via a linker group L, by the procedure described above, or to a X/X′ group via a linker group L′. The X/X′ attachment process demands that the R and X/X′ groups are derivatized with compatible precursors to L′. Suitable precursors to L′ are discussed above and the attachment process is well known for the one skilled in the art.

To give a compounds of general formula A-(B-L-R-(L-R-(L-R-(L-R-(L′-X′)ᵣ)ᵣ)ᵣ)ᵣ)ₙ, the chelator X can be transformed into a chelate X′ by complexation with a metal ion at any time of the synthesis.

Preferably the chelator X is transformed into a chelate X′ after the synthesis of the compound of formula A-(B-L-R-(L-R-(L-R-(L-R-(L′-X)ᵣ)ᵣ)ᵣ)ᵣ)ₙ.

The compounds of formula (II) and preferred embodiments thereof may be used as MR contrast agents. For this purpose, the compounds of formula (II) are formulated with conventional physiologically tolerable carriers like aqueous carriers, e.g. water and buffer solution and optionally excipients.

Hence in a further aspect the present invention provides a composition comprising a compound of formula (II) or preferred embodiments thereof and at least one physiologically tolerable carrier.

In a further aspect the invention provides a composition comprising a compound of formula (II) and preferred embodiments thereof and at least one physiologically tolerable carrier for use as MR imaging agent or MR spectroscopy agent.

To be used as agents for MR imaging or spectroscopy of the human or non-human animal body, said compositions need to be suitable for administration to said body. Suitably, the compounds of formula (II) or preferred embodiments thereof and optionally pharmaceutically acceptable excipients and additives may be suspended or dissolved in at least one physiologically tolerable carrier, e.g. water or buffer solutions. Suitable additives include for example physiologically compatible buffers like tromethamine hydrochloride, chelators such as DTPA, DTPA-BMA or compounds of formula (I) or preferred embodiments thereof, weak complexes of physiologically tolerable ions such as calcium chelates, e.g. calcium DTPA, CaNaDTPA-BMA, compounds of formula (I) or preferred embodiments thereof wherein X forms a complex with Ca²⁺ or CaNa salts of compounds of formula (I) or preferred embodiments thereof, calcium or sodium salts like calcium chloride, calcium ascorbate, calcium gluconate or calcium lactate. Excipients and additives are further described in e.g. WO-A-90/03804, EP-A-463644, EP-A-258616 and U.S. Pat. No. 5,876,695, the content of which are incorporated herein by reference.

Another aspect of the invention is the use of a composition comprising a compound of formula (II) or preferred embodiments thereof and at least one physiologically tolerable carrier as MR imaging agent or MR spectroscopy agent.

Yet another aspect of the invention is a method of MR imaging and/or MR spectroscopy wherein a composition comprising a compound of formula (II) or preferred embodiments thereof and at least one physiologically tolerable carrier is administered to a subject and the subject is subjected to an MR procedure wherein MR signals are detected from the subject or parts of the subject into which the composition distributes and optionally MR images and/or MR spectra are generated from the detected signals.

In a preferred embodiment, the subject is a living human or non-human animal body.

In a further preferred embodiment, the composition is administered in an amount which is contrast-enhancing effective, i.e. an amount which is suitable to enhance the contrast in the MR procedure.

In a preferred embodiment, the subject is a living human being or living non-human animal being and the method of MR imaging and/or MR spectroscopy is a method of MR tumour detection or a method of tumour delineation imaging.

In another embodiment, the subject is a living human or non-human animal being and the method of MR imaging and/or MR spectroscopy is a method of MR angiography, more preferred a method of MR peripheral angiography, renal angiography, supra aortic angiography, intercranial angiography or pulmonary angiography.

In another aspect, the invention provides a method of MR imaging and/or MR spectroscopy wherein a subject which had been previously administered with a composition comprising a compound of formula (II) or preferred embodiments thereof and at least one physiologically tolerable carrier is subjected to an MR procedure wherein MR signals are detected from the subject or parts of the subject into which the composition distributes and optionally MR images and/or MR spectra are generated from the detected signals.

The term “previously been administered” means that the method as described above does not contain an administration step of said composition to said subject. The administration of the composition has been carried out previous to the method as described above, i.e. before the method of MR imaging and/or MR spectroscopy according to the invention is commenced.

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. Compound of formula (II)
A-(B-L-R-(L-R-(L-R-(L-R-(L′-X′)ᵣ)ᵣ)ᵣ)ᵣ)ₙ (II)
wherein
A denotes a core;
B is the same or different and denotes a moiety that constitutes an obstacle for the rotation of the covalent bond between B and L.
L is the same or different and denotes a rigid linker moiety, wherein at least one L is present and under the proviso that L never links directly to another L;
L′ is present or not and if present is the same or different and denotes a linker moiety;
R is the same or different and denotes a branching moiety that reproduces with an individual multiplicity of r, wherein at least one R is present;
X′ is the same or different and denotes a paramagnetic tripodal chelate consisting of a tripodal chelator X and a paramagnetic metal ion M; and
r r is the same or different and denotes the integer 2, 3 or 4; and
n denotes an integer of 3 to 6.

2. Compound according to claim 1 wherein L is selected from 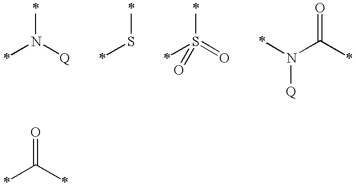
wherein
Q stands for H, C₁-C₈-alkyl, optionally substituted with one or more hydroxyl or amino groups; and
* stands for the possible attachment points to B and R

3. Compound according to claim 1 wherein A is a cyclic core, a carbon atom or an ethyl group.

4. Compound according to claim 1 wherein A is a saturated or non-saturated, aromatic or aliphatic ring comprising at least 3 carbon atoms and optionally one or more heteroatoms N, S or O, said ring being optionally substituted with one or more of the following substituents: C₁-C₃-alkyl, optionally substituted with hydroxyl or amino groups, amino or hydroxyl groups or halogen, provided that there are n attachment points left for groups B.

5. Compound according to claim 1 wherein B is a moiety whose rotation is hindered by sterical interaction with L.

6. Compound according to claim 1 wherein B comprises a benzene residue.

7. Compound according to claim 1 wherein the B moieties are interconnected by covalent bonds.

8. Compound according to claim 1 wherein R is selected from 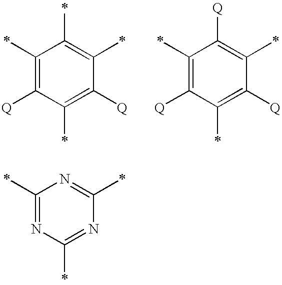
wherein
Q is H or CH₃ and;
* stands for the possible attachment points to L, L′ and X/X′.

9. Compound according to claim 1 wherein L′ is selected from 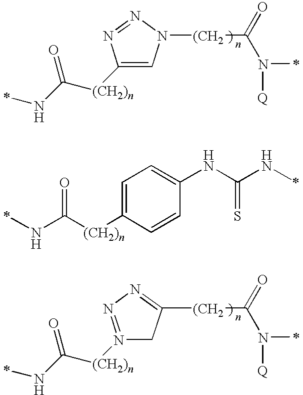
wherein
n is 0 to 6;
Q stands for H, C₁-C₈-alkyl, optionally substituted with one or more hydroxyl or amino groups; and
* stands for the possible attachment points to X/X′ and R

10. Compound according to claim 1 wherein M is a paramagnetic metal ion of atomic numbers 21 to 29, 42 to 44 or 57 to 71.

11. Compound according to claim 1 wherein X is of the general formula (IV): 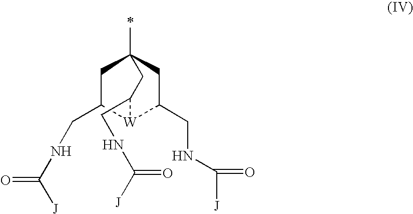
wherein
J is a chelator moiety consisting of a 6-membered aromatic or partially saturated ring system containing up to three heteroatoms selected from nitrogen and oxygen and having a hydroxyl group as a first substituent bound to a first atom in said ring system, and a hydroxyl group or an oxygen atom doubly bound to a second atom in said ring system wherein said first and second atom are adjacent atoms and wherein said first and second substituents are in ring positions such that J is capable of forming a complex with a paramagnetic metal ion; and wherein J is optionally substituted by up to three additional substituents, R¹, where each R¹ is independently a hydrophilic group which renders the compounds of formulae (III) and (IV) soluble in aqueous solutions;
* stands for the attachment point to L′ or R; and
W and the bonds represented as dotted lines are present or absent and when present, W is N.

12. Compound according to claim 1 wherein X is of the general formula (III): 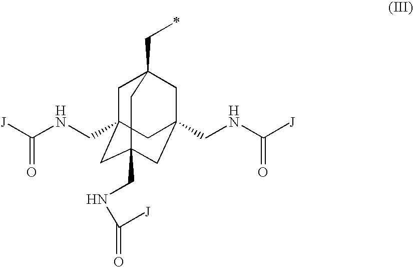
wherein
J is a chelator moiety consisting of a 6-membered aromatic or partially saturated ring system containing up to three heteroatoms selected from nitrogen and oxygen and having a hydroxyl group as a first substituent bound to a first atom in said ring system, and a hydroxyl group or an oxygen atom doubly bound to a second atom in said ring system wherein said first and second atom are adjacent atoms and wherein said first and second substituents are in ring positions such that J is capable of forming a complex with a paramagnetic metal ion; and wherein J is optionally substituted by up to three additional substituents, R¹, where each R¹ is independently a hydrophilic group which renders the compounds of formulae (III) and (IV) soluble in aqueous solutions;
* stands for the attachment point to L′ or R; and
W and the bonds represented as dotted lines are present or absent and when present, W is N.

13. Composition comprising the compound according to claim 1 and at least one physiologically tolerable carrier.

14. Composition according to claim 13 for use as MR imaging agent or MR spectroscopy agent.

15. Use of the composition according to claim 13 as MR imaging contrast agent or MR spectroscopy agent.

16. Method of MR imaging and/or MR spectroscopy wherein the composition according to claim 13 is administered to a subject and the subject is subjected to an MR procedure wherein MR signals are detected from the subject or parts of the subject into which the composition distributes and optionally MR images and/or MR spectra are generated from the detected signals.

17. Method of MR imaging and/or MR spectroscopy wherein a subject which had been previously administered with the composition according to claim 13 is subjected to an MR procedure wherein MR signals are detected from the subject or parts of the subject into which the composition distributes and optionally MR images and/or MR spectra are generated from the detected signals.

18. Method for the preparation of a compound according to claim 1 comprising
a) using as a first building block a precursor to core A that is substituted with reactive groups which allow for the attachment of moieties B;
b) reacting moieties B or precursors thereof with said first building block to form a second building block consisting of the core A and B;
c) reacting R or a precursor thereof with said second building block to form a linker L and a third building block consisting of A, B, L and R;
d) optionally sequentially reacting further Rs or precursors thereof with said third building block to form further Ls and a fourth building block;
e) reacting X or a precursor thereof with said third or fourth building block to form a linker L′ and a fifth building block consisting of A, B, Lₙ, Rₙ, L′ and X; and
f) complexing said fifth building block with a paramagnetic metal ion M.
